(19) United States
(12) Patent Application Publication
(10) Pub. No.: US 2023/0270773 A1
(43) Pub. Date: Aug. 31, 2023

(54) METHODS OF TREATING CANCER WITH A STING AGONIST

(71) Applicants: Immunesensor Therapeutics, Inc., Dallas, TX (US); The Board of Regents of the University of Texas System, Austin, TX (US)

(72) Inventors: Zhijian CHEN, Dallas, TX (US); Lijun SUN, Dallas, TX (US)

(21) Appl. No.: 17/768,922

(22) PCT Filed: Oct. 14, 2020
(86) PCT No.: PCT/US2020/055633

§ 371 Date: Apr. 14, 2022

Related U.S. Application Data
(60) Provisional application No. 62/914,881, filed on Oct. 14, 2019.

(51) Int. Cl. A61K 31/708; C07K 16/28; A61P 35/00

(52) U.S. Cl. CPC A61K 31/708; A61P 35/00; C07K 16/2818; C07K 16/2827

(57) ABSTRACT

The disclosure provides methods of treating a cancer in a patient by administering a compound (“Compound A”) having the following structure or a pharmaceutically acceptable salt thereof: at particular dosing regimens and, optionally, in combination with one or more compounds that inhibit immune checkpoint proteins.

### 1. FIELD

This disclosure pertains to, among other things, the use of a particular STING agonist either alone or in combination with an immune checkpoint inhibitor for activating the immune system to treat cancer.

### 2. BACKGROUND

The treatment of advanced solid tumor malignancies as well as many hematologic malignancies continues to be defined by high unmet medical need. In most settings, treatment with cytotoxic chemotherapy and targeted kinase inhibitors leads to the emergence of drug-resistant tumor clones and subsequent tumor progression and metastasis.

In recent years, notable success has been achieved through alternate approaches oriented around activation of immune-mediated tumor destruction. The immune system plays a pivotal role in defending humans and animals against cancer. The anti-tumor effect is controlled by positive factors that activate anti-tumor immunity and negative factors that inhibit the immune system. Negative factors that inhibit anti-tumor immunity include immune checkpoint proteins, such as cytotoxic T-lymphocyte-associated protein 4 (CTLA-4), programmed cell death-1 (PD-1), and programmed death-ligand 1 (PD-L1). Immuno-oncology (IO) approaches, including antibodies against these checkpoint proteins, have shown remarkable efficacy in several types of human cancers.

However, existing cancer immunotherapy through immune checkpoint blockade is effective for only a small fraction (on average 20-30%) of cancer patients. The patients who are refractory to immune checkpoint blockade often have tumors that are not inflamed, or so-called “cold” tumor cells, i.e., they lack tumor-infiltrating leukocytes (TILs), such as cluster of differentiation 8 (CD8) T cells, or the tumor microenvironment suppresses the functions of the TILs. A major thrust of ongoing cancer drug development research remains focused on transforming “cold” tumor cells into “hot” tumor cells in order to achieve better tumor control across a wider array of patients.

The innate immune system, which is the first line of defense against pathogens and cancer cells, is important for turning the non-inflamed tumors (“cold”) into an inflamed (“hot”) microenvironment. A recently discovered innate immunity pathway, the Cyclic GMP-AMP Synthase (cGAS)-Stimulator of Interferon Genes (STING) pathway, plays a critical role in anti-tumor immunity. cGAS is a DNA sensing enzyme that activates the type-I interferon pathway. Upon binding DNA, cGAS is activated to synthesize 2′3′-cyclic-GMP-AMP (2′3′-cGAMP), which then functions as a secondary messenger that binds to and activates the adaptor protein STING. STING then activates a signal transduction cascade leading to the production of type-I interferons, cytokines and other immune mediators.

While cytokine production is essential for generating anti-tumor immunity, high cytokines levels pose a safety concern. Specifically, high cytokine levels can evoke an inflammatory response in cancer patients undergoing immunotherapy. The inflammatory response can be enhanced in the presence of other compounds that modulate the immune system, for instance, immune checkpoint inhibitors. Therefore, developing toxicologically acceptable anti-tumor immunotherapies to treat cancer is an important goal in need of further advancement.

### 3. SUMMARY

The disclosure provides methods of treating cancer patients comprising administering a compound (“Compound A”) having the following structure, or a pharmaceutically acceptable salt thereof:

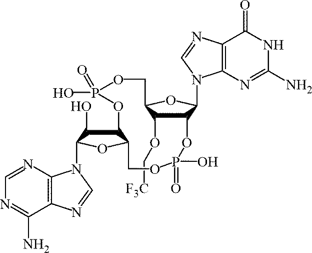

at particular dosing regimens, either alone or in combination with one or more compounds that inhibits immune checkpoint proteins. Compound A is a cyclic dinucleotide that is capable of activating STING and was described in U.S. Published Application No. 2018/0230177, which is incorporated herein by reference. Various salt forms of Compound A can be administered to a cancer patient. For instance, in one embodiment, a therapeutically effective amount of a sodium salt of Compound A is administered to the cancer patient. It will be understood that any reference to Compound A in the disclosure also includes pharmaceutically acceptable salts thereof.

It has been discovered that Compound A can act both locally and systemically to exert a powerful ant-tumor effect. Compound A, when administered at particular dosages to a cancer patient in need thereof, is capable of substantially reducing or preventing the spreading of metastasis. The ability of Compound A to reduce or prevent the onset and/or progression of metastasis can be potentiated when administered together with an immune checkpoint inhibitor, particularly a PD-L1 or PD-1 inhibitor. Additionally, it has been discovered that Compound A exerts a powerful abscopal effect when administered alone or in combination with an immune checkpoint inhibitor, particularly a PD-L1 or PD-1 inhibitor.

In one aspect, the disclosure provides methods of treating metastasis in a human cancer patient comprising administering a therapeutically effective amount of Compound A to the patient. In certain embodiments, Compound A can be administered intratumorally. In other embodiments, Compound A can be administered systemically. For instance, in particular embodiments, Compound A can be administered subcutaneously, intramuscularly or intravenously.

In one aspect, the disclosure provides methods of preventing metastasis in a human cancer patient comprising administering a therapeutically effective amount of Compound A to the patient. In certain embodiments, Compound A can be administered intratumorally. In other embodiments, Compound A can be administered systemically. For instance, in particular embodiments, Compound A can be administered subcutaneously, intramuscularly or intravenously.

In one embodiment, the disclosure provides methods of treating or preventing metastasis in a human cancer patient comprising administering a therapeutically effective amount of Compound A to the patient in combination with one or more immune checkpoint inhibitors. In certain embodiments, the patient has already undergone at least one cycle of treatment with the one or more immune checkpoint inhibitors prior to the start of administration of Compound A. In certain embodiments, Compound A is administered prior to or concurrently with the administration of the one or more immune checkpoint inhibitors. In some embodiments, the immune checkpoint inhibitor is a PD-1 inhibitor or a PD-L1 inhibitor. In some embodiments, the immune checkpoint inhibitor is a CTLA4 inhibitor. In other embodiments, compound A is administered prior to or concurrently with the administration of the CTLA4 inhibitor and a PD-1 inhibitor or a PD-L1 inhibitor.

In some embodiments of the disclosure, Compound A can be combined with an immune checkpoint inhibitor to treat cancers that are resistant or refractory to immune checkpoint therapy. For instance, Compound A can be used to treat primary or metastasizing tumors that are resistant to immune checkpoint therapy. Compound A can be administered simultaneously, prior to, or following administration of the immune checkpoint inhibitor, such as simultaneously, prior to, or following administration of a treatment cycle of the one or more immune checkpoint inhibitors. In some embodiments, the cancer is resistant to treatment with immune checkpoint inhibitors when administered in the absence of Compound A. In some embodiments, the immune checkpoint inhibitor inhibits the interaction between PD-L1 and PD-1. For instance, the immune checkpoint inhibitor can be an antagonist of PD-L1 (e.g., a PD-L1 antibody) or an antagonist of PD-1 (e.g., a PD-1 antibody).

It has been found that in certain tumors that are refractory to immune checkpoint inhibition, Compound A can be administered together with the immune checkpoint inhibitor at doses that are less than doses required to treat the patient when Compound A is administered as a monotherapy. As a result, the combination therapy can be administered without evoking a harmful inflammatory response in the patient that might be evoked by the higher dose of Compound A when used as a monotherapy.

The disclosure also provides particular dosing regimens for administration of Compound A either by itself (i.e., monotherapy) or together with an immune checkpoint inhibitor to a human cancer patient in need thereof. The dosing regimens disclosed herein are capable of evoking a powerful anti-tumor effect without concurrent side effects often associated with excessive cytokine production. Surprisingly, it has been discovered that Compound A shows a profound anti-tumor effect even at dosage levels where it induces low levels of cytokine production. Therefore, when dosed using particular dosing amounts and regimens described herein, Compound A can be administered with minimal side effects while still showing a significant anti-tumor effect.

In a particular embodiment, the disclosure provides methods of treating a cancer patient comprising administering multiple cycles of Compound A to the patient, wherein the first cycle comprises administering Compound A on days 1, 8, and 15 of a four-week period, and subsequent cycles comprise administering Compound A on days 1 and 15 (i.e., biweekly) of a four-week period. Compound A can be administered intratumorally or systemically, including subcutaneously, intramuscularly or intravenously. In some embodiments, on days of the cycle designated for administration, Compound A can be administered at a dosage in the range of 50 µg to 6,500 µg. In some embodiments, on days of the cycle designated for administration, Compound A can be administered at a dosage in the range of 100 µg to 3,000 µg. In some embodiments, on days of the cycle designated for administration, Compound A can be administered at a dosage in the range of 100 µg to 1,200 µg.

In another embodiment, the disclosure provides methods of treating cancer, comprising administering to a cancer patient a dosing regimen comprising a priming dose of Compound A at the onset of the therapy, followed by administration of maintenance doses of Compound A. For instance, the priming dose can be administered on day 1 of a treatment cycle and the maintenance doses can be administered thereafter starting on day 2, 3, 4, 5, 6, 7, 8, 9, 10, or 11 of the treatment cycle. In some embodiments, the dosing regimen also involves administration of an immune checkpoint inhibitor. The immune checkpoint inhibitor can be administered together with the priming dose of Compound A or following administration of the priming dose, such as after the priming dose but before the maintenance doses, concurrently with the maintenance doses, or following a treatment cycle of the maintenance doses.

In one embodiment, Compound A is administered to a human cancer patient already receiving immune checkpoint inhibition therapy, such as for whom the cancer has stabilized. In particular embodiments, the cancer patient has undergone at least 1 or 2 cycles of immune checkpoint inhibitor therapy prior to administration of Compound A. For instance, the cancer patient may have undergone 2, 3, 4, 5, 6, 7, or 8 cycles of immune checkpoint inhibition therapy prior to administration of Compound A. In certain of these embodiments, the cancer patient continues to receive immune checkpoint inhibition therapy with successive cycles as Compound A is administered.

In another aspect, the disclosure provides a method of treating a cancer in a patient, comprising intratumorally administering to the patient a dose of Compound A at a site that is accessible for intratumoral administration, wherein some occurrences of the cancer are inaccessible for intratumoral administration and wherein the dose provides sufficient cytokine activation to promote an immune response by the patient against the inaccessible occurrences of the cancer. In some embodiments, Compound A is administered in combination with an immune checkpoint inhibitor.

### 4. BRIEF DESCRIPTION OF THE FIGURES

FIG. 1 shows blood levels of compound and IFNβ after Compound A was administration to mice. Panel A of FIG. 1 shows the blood level of Compound A after mice were dosed with 5 mg/kg of Compound A. Panel B of FIG. 1 shows the blood level of IFNβ after mice were dosed with 5 mg/kg of Compound A. Panel C of FIG. 1 shows the blood levels of IFNβ after mice were dosed with 0, 0.15, 0.50, 1.50, 5, 15, and 50 mg/kg of Compound A. Data were presented as mean ±SD of 3 mice per group. IM: intramuscular; IV: intravenous; SC: subcutaneous.

FIG. 2 shows IFNβ, TNFα, and interleukin 6 (IL-6) production induced by Compound A at various concentrations in human peripheral blood mononuclear cells (PBMCs). Panel A of FIG. 2 shows IFNβ production induced by Compound A at various concentrations. Panel B of FIG. 2 shows TNFα production induced by Compound A at various concentrations. Panel C of FIG. 2 shows IL-6 production induced by Compound A at various concentrations.

FIG. 3 shows the results of studies of Compound A on B16 melanoma. Panels A-C of FIG. 3 show the tumor shrinking effect of intratumoral administration of Compound A (administered alone or in combination with a PD-L1 antibody) on B16 melanoma at various concentrations. Panel D of FIG. 3 shows the statistics of tumor growth curves. Numbers indicate p values between each comparison groups obtained using two-way ANOVA (analysis of variance). Panels E-F of FIG. 3 show survival of mice following intratumoral administration of Compound A (administered alone or in combination with a PD-L1 antibody) on B16 melanoma at various concentrations. Panel H of FIG. 3 shows the statistics on the survival curves. Numbers indicate p values between each comparison groups obtained using log rank (Mantel Cox) test.

FIG. 4 shows the results of studies of Compound A on AG104A fibrosarcoma. Panel A of FIG. 4 shows the tumor shrinking effect of subcutaneous administration of Compound A (administered alone or in combination with a PD-L1 antibody) on an AG104A (fibrosarcoma) model. Panel B of FIG. 4 shows the percent survival of mice following subcutaneous administration of Compound A (administered alone or in combination with a PD-L1 antibody) on an AG104A (fibrosarcoma) model. Panel C of FIG. 4 shows Statistical analysis of tumor growth data up to Day 19. P values between comparison groups were calculated using two-way ANOVA. Panel D of FIG. 4 shows Statistical analysis of survival data. P values between comparison groups were obtained using log rank (Mantel Cox) test.

FIG. 5 shows the effects of subcutaneous administration of Compound A on AG104LD fibrosarcoma. Groups of B6C3F 1 mice bearing AG104LD fibrosarcoma were treated with anti-PDL1 antibody, Compound A, or combination of both, at day 4, 7, 11, 14, 18, and day 21. Each group contained 5 mice. Panel A of FIG. 5 shows tumor growth over time. Data are shown as mean± SEM. Panel B of FIG. 5 shows survival of the mice over time. Panel C of FIG. 5 shows statistical analysis of tumor growth data. P values between comparison groups were obtained using two-way ANOVA. Panel D of FIG. 5 shows statistical analysis of mice survival data. P values between comparison groups were obtained using log rank (Mantel Cox) test.

FIG. 6 shows the effect of intratumoral administration of Compound A on LL2 tumors. Groups of C57BL6 mice bearing LL2 tumors were treated with anti-PDL1 antibody, Compound A, or a combination of both, on days 5, 10, 14, and 17. Each group contained 5 mice. Panel A of FIG. 6 shows tumor growth over time. Data are shown as mean±SEM. Panel B of FIG. 6 shows survival of the mice over time. Panel C of FIG. 6 shows statistical analysis of tumor growth data. P values between comparison groups were obtained using two-way ANOVA. Panel D of FIG. 6 shows statistical analysis of mice survival data. P values between comparison groups were obtained using log rank (Mantel Cox) test.

FIG. 7 shows the effect of intratumoral administration of Compound A on 4T1 tumors. Groups of BALB/c mice bearing 4T1 tumors were treated with anti-PDL1 antibody, Compound A, or combination of both, at day 5, 8, 11, and 15. Each group contained 5 mice. Panel A of FIG. 7 shows tumor growth over time. Data are shown as mean± SEM. Panel B of FIG. 7 shows survival of the mice over time. Panel C of FIG. 7 shows statistical analysis of tumor growth data. P values between comparison groups were obtained using two-way ANOVA. Panel D of FIG. 7 shows statistical analysis of mice survival data. P values between comparison groups were obtained using log rank (Mantel Cox) test.

FIG. 8 shows the abscopal effect of Compound A on B16 melanoma. Mice bearing B16 melanoma on both left and right flanks were treated intratumorally on their right sites (primary) with indicated doses of Compound A, at day 5, 8, 11, and 15. Tumors on left sites (distant) were left untreated. Each group contains 7 mice. Panel A of FIG. 8 shows primary tumor growth over time. Panel B of FIG. 8 shows distant tumor growth over time. Panel C of FIG. 8 shows statistical analysis of tumor growth data in Panel A. P values between comparison groups were obtained using two-way ANOVA. Panel D of FIG. 8 shows statistical analysis of tumor growth data in Panel B. P values between comparison groups were obtained using two-way ANOVA. Panel E of FIG. 8 shows mice survival over time. Panel F of FIG. 8 shows statistical analysis of mice survival data in Panel E. P values between comparison groups were obtained using log rank (Mantel Cox) test.

FIG. 9 shows the abscopal effect of Compound A on AG104A fibrosarcoma. C3B6F1 mice (N=8) bearing AG104A tumors on both left and right flanks were treated intratumorally on the right flank (primary) with Compound A on Days 5, 8, 11, and 15 after tumor inoculation. Tumors on the left flank (distant) were not treated. Panel A of FIG. 9 shows primary tumor growth over time. Panel B of FIG. 9 shows distant tumor growth over time. Panel C of FIG. 9 shows statistical analysis of tumor growth data in A. P values between comparison groups were obtained using a two-way ANOVA. Panel D of FIG. 9 shows statistical analysis of tumor growth data in Panel B. P values between comparison groups were obtained using a two-way ANOVA. Panel E of FIG. 9 shows percent survival over time. Panel F of FIG. 9 shows statistical analysis of survival data in E. P values between comparison groups were obtained using log rank (Mantel Cox) test.

FIG. 10 shows the effect of Compound A on tumor metastasis using a B16 melanoma lung metastasis mouse model. Panel A of FIG. 10 shows the growth curves of primary tumors over time. Panel B of FIG. 10 shows statistical analysis of tumor growth data in A. P values between comparison groups were obtained using two-way ANOVA. Panel C of FIG. 10 shows images showing lung metastasis in all groups. The right two panels show magnified images of lungs representing selected treatment groups. Panel D of FIG. 10 shows quantification of lung metastases in Panel C. Each symbol represents one mouse. Panel E of FIG. 10 shows statistical analysis of lung metastasis in Panel D. P values between comparison groups were obtained using unpaired t-test.

FIG. 11 shows the effect of a triple combination of Compound A (I.T.), PD-L1 antibody (I.P.), and CTLA4 antibody (I.P.). Groups of C57BL6 (n=5) bearing B16F10 tumors were treated as indicated on day 6, 10 and 14 after tumor implantation. Panel A of FIG. 11 shows tumor growth over time, and Panel B of FIG. 11 shows mice survival over time. Data are shown as mean ±SEM.

### 5. DETAILED DESCRIPTION

### 5.1. Methods of Treatment

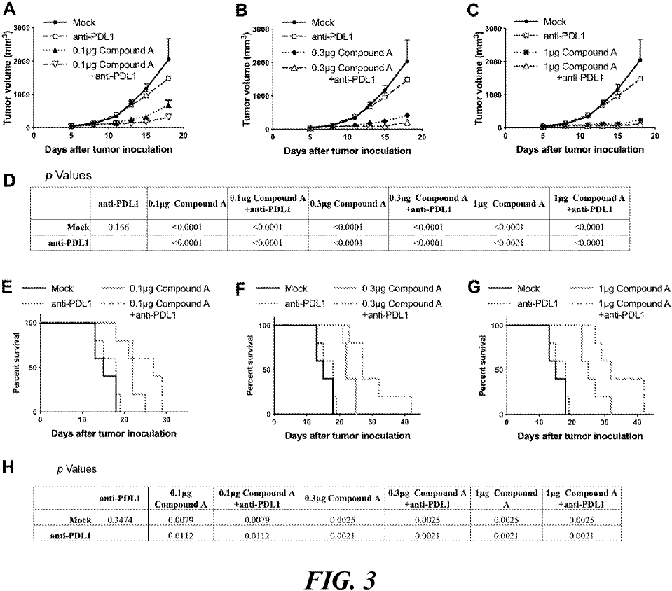

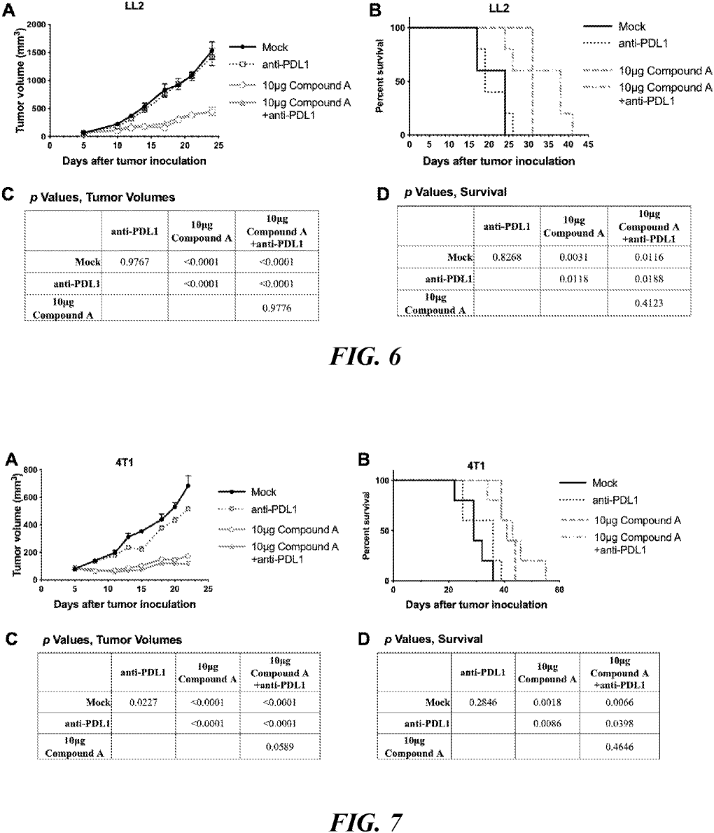

Compound A is used to treat cancer. In accordance with the disclosure, Compound A can be used to treat both primary tumors and metastasizing tumors. In some embodiments, Compound A can be administered at dosage levels or under a particular dosing regimen as disclosed herein that results in shrinking or eradicating primary tumors and developing metastases stemming from the primary tumors. Compound A may also prevent the formation of metastasis if administered prior to the tumor spreading from a tissue to other parts of the body. As shown in Examples 5-9 and FIGS. 3-10, administration of Compound A is highly efficacious in eradicating both primary tumors and developing metastases.

Accordingly, in one aspect, the disclosure provides methods of treating cancer in a subject comprising administering a pharmaceutical composition comprising a pharmaceutically acceptable amount of Compound A. In another aspect, the disclosure provides methods of treating cancer in a subject comprising administering a pharmaceutical composition comprising a pharmaceutically acceptable amount of Compound A. In some embodiments, the pharmaceutical compositions are administered to mammals in need thereof. In particular embodiments, the pharmaceutical compositions are administered to a human patient in need thereof.

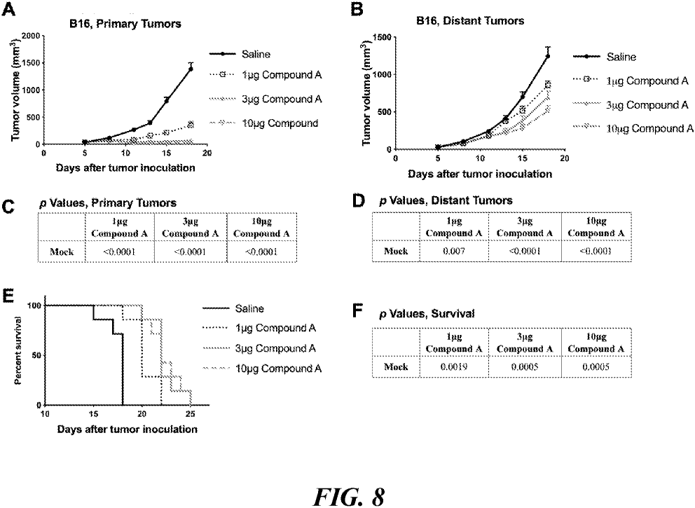

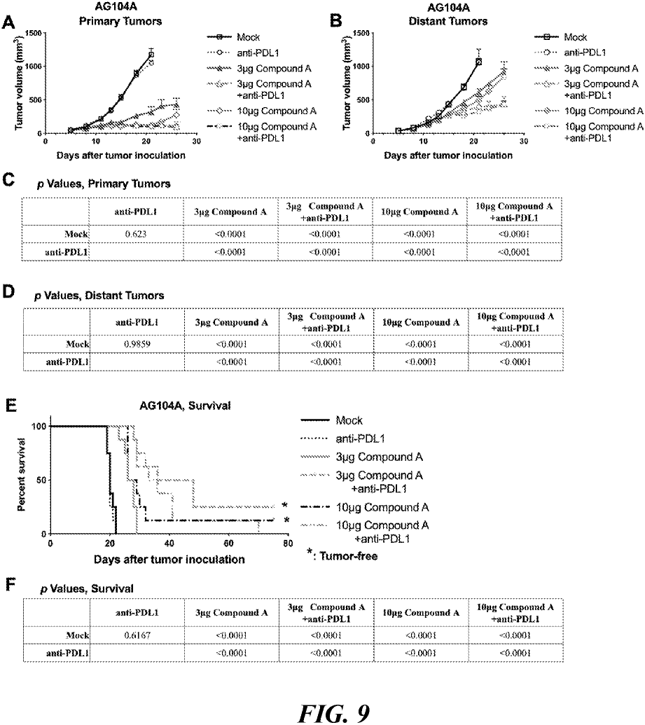

In some embodiments, Compound A is administered intratumorally into the primary tumor of the patient. It has been found that when Compound A is administered intratumorally into the primary tumor, tumor growth is suppressed not only at the site of the primary tumor, but also at the site of distant tumors (see Example 8 and FIGS. 8 and 9). Therefore, Compound A displays a profound abscopal effect. Accordingly, the disclosure provides methods of treating both primary and distant tumors (including accessible and inaccessible cancers) by administering Compound A at dosage levels or at a particular dosing regimen disclosed herein.

In some embodiments, Compound A is administered intratumorally to a cancer patient at a site that is accessible for intratumoral administration, wherein some occurrences of the cancer are inaccessible for intratumoral administration, and wherein the dose provides sufficient cytokine activation to promote an immune response by the patient against the inaccessible occurrences of the cancer. The inaccessible occurrences of the cancer may be tumor masses or developing metastases that cannot be easily accessed by intratumoral administration. In some embodiments, the tumors are not amenable to removal by surgery. In particular embodiments, Compound A is administered in combination with an immune checkpoint inhibitor, as discussed below.

In other embodiments, Compound A is administered systemically. For instance, Compound A can be administered intravenously, intramuscularly, or subcutaneously to a cancer patient. Pharmacokinetic studies (see Example 1) show that Compound A is highly bioavailable following either subcutaneous or intratumoral administration. Accordingly, Compound A is efficacious, even following systemic administration. Moreover, the effect of Compound A on shrinking distant tumors and eradicating metastases may be explained, in part, by the systemic availability of Compound A.

In particular embodiments, Compound A can be used to treat cancers of the lung, bone, pancreas, skin, head, neck, uterus, ovaries, stomach, colon, breast, esophagus, small intestine, bowel, endocrine system, thyroid gland, parathyroid gland, adrenal gland, urethra, prostate, penis, testes, ureter, bladder, kidney, or liver. Further cancers treatable by Compound A include rectal cancer; cancer of the anal region; carcinomas of the fallopian tubes, endometrium, cervix, vagina, vulva, renal pelvis, and renal cell; sarcoma of soft tissue; myxoma; rhabdomyoma; fibroma; lipoma; teratoma; cholangiocarcinoma; hepatoblastoma; angiosarcoma; hemagioma; hepatoma; fibrosarcoma; chondrosarcoma; myeloma; chronic or acute leukemia; lymphocytic lymphomas; primary CNS lymphoma; neoplasms of the CNS; spinal axis tumors; squamous cell carcinomas; synovial sarcoma; malignant pleural mesotheliomas; brain stem glioma; pituitary adenoma; bronchial adenoma; chondromatous hanlartoma; inesothelioma; Hodgkin’s Disease; or a combination of one or more of the foregoing cancers.

In particular embodiments, Compound A can be used to treat a cancer that is refractory or unresponsive to immune checkpoint inhibitory therapy. Such cancers may include but are not limited to prostate cancer, pancreatic cancer, lymphoma, head and neck cancer, kidney cancer, melanoma, colon cancer, breast cancer, and lung cancer. In certain embodiments, the cancer is selected from prostate cancer, pancreatic cancer, lymphoma, head and neck cancer, and kidney cancer. In some embodiments, the cancer is selected from melanoma, colon cancer, breast cancer, and lung cancer. As described herein, in such refractory or unresponsive tumors, Compound A can synergize with the immune checkpoint inhibitor therapy to produce a potent anti-tumor response.

### 5.2. Combination Therapy

In another aspect, the disclosure provides methods of treating cancer in a subject by administering a pharmaceutical composition comprising a pharmaceutically acceptable amount of Compound A with at least one additional anti-cancer agent to a subject (e.g., a human). Compound A and the one or more additional anti-cancer agents may be administered together or separately and, when administered separately, administration may occur simultaneously or sequentially, in any order, by any convenient route in separate or combined pharmaceutical compositions. The amounts of Compound A and the other pharmaceutically active anti-cancer agent(s) and the relative timings of administration will be selected in order to achieve the desired combined therapeutic effect.

The combination of Compound A and one or more anti-cancer agents may be administered together in a single pharmaceutical composition. Alternatively, Compound A and the one or more anti-cancer agents may be formulated separately. When formulated separately they may be provided in any convenient composition, conveniently, in such a manner as known for such compounds in the art.

Accordingly, Compound A may be employed with other therapeutic methods of cancer treatment, e.g., in anti-neoplastic therapy, combination therapy with immune checkpoint inhibitors, other chemotherapeutic, hormonal, antibody agents as well as surgical and/or radiation treatments.

In one embodiment, Compound A is employed in combination with an immune checkpoint inhibitor to treat cancer. Immune checkpoint inhibitors, such as humanized antibodies against PD-1, PD-L1, and CTLA4, have shown to be highly successful in treating several types of metastatic cancer, including melanoma, non-small cell lung cancers, renal cell carcinoma and bladder cancer (Sharma and Allison, 2015, Science 348, 56). However, still only a small percentage of cancer patients benefit from the checkpoint inhibitor therapies, in part because insufficient number of anti-tumor immune cells, such as CD8 T cells, are generated and/or infiltrated into the tumors. As shown in examples described herein, the combination of Compound A and an immune checkpoint inhibitor is capable of functioning synergistically to treat cancers that are refractory to monotherapy with the immune checkpoint inhibitor.

In particular embodiments, Compound A and the immune checkpoint inhibitor are administered to a cancer patient who has previously undergone treatment with the immune checkpoint inhibitor.

In other embodiments, Compound A and the immune checkpoint inhibitor are administered to a cancer patient that is unresponsive to therapy by the immune checkpoint inhibitor administered in the absence of Compound A. In such embodiments, the immune checkpoint inhibitor when administered in the absence of Compound A, is unable to slow or stop the growth (progression) of the tumor or to reduce the level of a particular tumor biomarker associated with the cancer being treated. For instance, Compound A can be used to treat primary or metastasizing tumors that are refractory to immune checkpoint therapy or even completely resistant to immune checkpoint therapy. As shown in the examples, Compound A is capable of rendering these resistant cancers susceptible to immune checkpoint therapy. As shown in FIGS. 3-10, tumors that showed a limited response and tumors that were completely nonresponsive to PD-L1 inhibition were treatable with Compound A. Surprisingly, for these tumors that were not responsive to PD-L1 inhibition, administration of the PD-L1 inhibitor potentiated the anti-tumor effect of Compound A.

Moreover, as shown in Examples 6-9, it has been found that in certain tumors that are refractory to immune checkpoint inhibition, Compound A can be administered together with the immune checkpoint inhibitor at dosages that are less than doses of Compound A that are required to treat the patient when Compound A, is administered as a monotherapy. For instance, if the immune checkpoint is administered in accordance with the dosing schedule reflected on its product label, then Compound A can be administered at dosage levels that are generally less than dosage levels that are required to evoke an anti-tumor response when Compound A is administered as a monotherapy. In some embodiments, the dosage of Compound A, when used in combination with an immune checkpoint inhibitor, will be from 1.2-fold to 3-fold less than dosage that is required to evoke an anti-tumor response when Compound A is administered as a monotherapy. In some instances, when Compound A is administered in combination with a PD-L1 inhibitor (see FIG. 9), smaller amounts of Compound A were shown to be equally or even more efficacious than larger amounts of Compound A. As a result, the combination therapy can be administered without evoking a severe inflammatory response in the patient.

In some embodiments, Compound A can be administered together with a PD-L1 inhibitor, a PD-1 inhibitor, or a CTLA-4 inhibitor or a combination thereof. For example, Compound A can be administered together with both a PD-L1 inhibitor and a CTLA-4 inhibitor, with both a PD-1 inhibitor and a CTLA-4 inhibitor, or with both a PD-L1 inhibitor and a PD-1 inhibitor.

Examples of PD-L1 inhibitors that can be used in combination with Compound A include, but are not limited to, atezolizumab (Tecentriq®), avelumab (Bavencio®), durvalumab (Imfinzi®), BMS-936559, and CK-301.

Examples of PD-1 inhibitors that can be used in combination with Compound A include, but are not limited to, pembrolizumab (Keytruda®), nivolumab (Opdivo®), cemiplimab (Libtayo®), AMP-224, AMP-514, and PDR001.

Examples of CTLA-4 inhibitors that can be used in combination with Compound A include, but are not limited to, ipilimumab (Yervoy®) and tremelimumab.

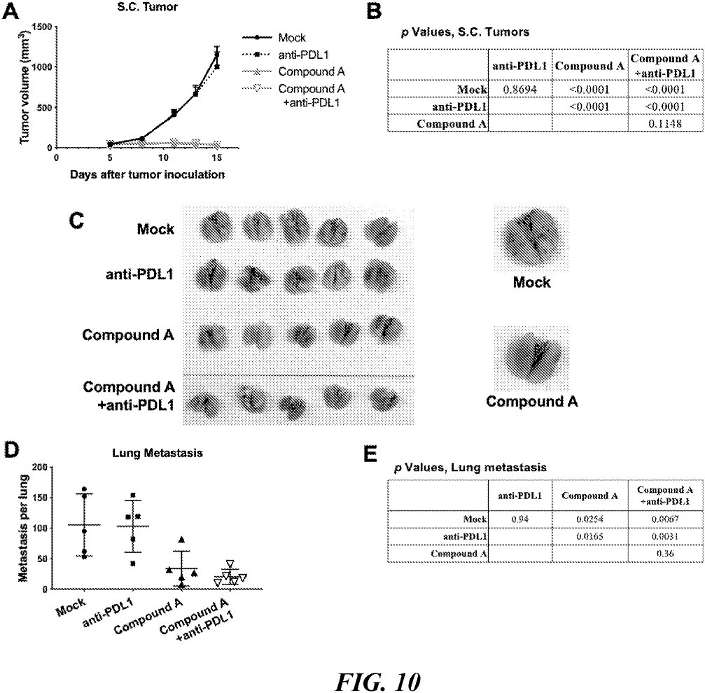

The anti-tumor effect of Compound A is enhanced when administered to a patient undergoing or who has undergone therapy with an immune checkpoint inhibitor. As shown in Example 9 and FIG. 10, the ability of Compound A to reduce or eliminate metastases is also potentiated by administration of an immune checkpoint inhibitor. Likewise, as shown in Example 8 and FIG. 9, the abscopal effect demonstrated by administration of Compound A at the site of a primary tumor is potentiated by administration of an immune checkpoint inhibitor. Therefore, the combination of Compound A and an immune checkpoint inhibitor is particularly efficacious in treating advanced tumors, particularly metastasizing tumors or tumors that have established in secondary organs of the body.

In certain embodiments, the patient has already undergone at least one cycle of treatment with the one or more immune checkpoint inhibitors. In particular, for a commercially approved immune checkpoint inhibitor (e.g., a PD-1 or PD-L1 inhibitor), the patient has undergone at least one dosing cycle in accordance with the approved label of the immune checkpoint inhibitor. In some embodiments, prior to administration of Compound A, the patient has already undergone from 2 to 20 cycles of treatment with the immune checkpoint inhibitor. In some embodiments, Compound A is administered to a cancer patient undergoing immune checkpoint inhibitor therapy after the disease has stabilized. In some embodiments, Compound A is administered to a cancer patient after the patient’s cancer has grown refractory to the immune checkpoint inhibitor. For patients with stabilized disease or tumors that have grown refractory to immune checkpoint inhibitor therapy, the immune checkpoint inhibitor can still be administered to the patient after treatment of Compound A commences.

In other embodiments, Compound A is administered to the cancer patient prior to the patient receiving an immune checkpoint inhibitor (e.g., a PD-1 inhibitor or PD-L1 inhibitor). For instance, the patient may receive from 1- to 10-dosing cycles of Compound A, as disclosed herein, prior to receiving the immune checkpoint inhibitor. In some such embodiments, administration of Compound A would continue after administration with the immune checkpoint inhibitor commences. In other such embodiments, administration with the immune checkpoint inhibitor would stop when administration with the immune checkpoint inhibitor commences.

In some embodiments, Compound A can be administered during the same time as administration of the immune checkpoint inhibitor (e.g., a PD-1 inhibitor or PD-L1 inhibitor). For instance, Compound A and the immune checkpoint inhibitor can both be administered in the same dosing cycles. In some such embodiments, the cancer patient previously received neither therapy with Compound A nor the immune checkpoint inhibitor.

In one embodiment, Compound A is administered in combination with a PD-1 or PD-L1 inhibitor to treat a metastasizing tumor. Compound A can be administered prior to, concurrently or after treatment with the immune checkpoint inhibitor. In certain embodiments, the immune checkpoint inhibitor can be administered according to a dosing cycle as disclosed herein. In such embodiments, the PD-1 or PD-L1 inhibitor can be administered on the same day or on different days of the dosing cycle. If the PD-1 or PD-L1 inhibitor is a commercial product, then the PD-1 or PD-L1 inhibitor may be administered in accordance with the label of the product to be administered. The amount of PD-1 or PD-L1 inhibitor administered to the patient can be the amount reflected on the product label. In certain embodiments, the amount of the PD-1 or PD-L1 inhibitor administered to the patient can be less than the amount reflected on the product label.

In another embodiment, Compound A is administered in combination with a PD-1 or PD-L1 inhibitor to treat a tumor that is not readily accessible by intratumoral administration. Compound A can be administered intratumorally in a tissue that is remote from the inaccessible tumor in order to induce an abscopal effect. In such cases, the tumor receiving the intratumorally administered dose of Compound A can be a primary tumor or a secondary tumor.

In another embodiment, Compound A is administered in combination with a CTLA4 inhibitor to treat a tumor that is not readily accessible by intratumoral administration. Compound A can be administered intratumorally in a tissue that is remote from the inaccessible tumor in order to induce an abscopal effect. In such cases, the tumor receiving the intratumorally administered dose of Compound A can be a primary tumor or a secondary tumor.

In another embodiment, Compound A is administered in combination with a PD-1 inhibitor (or a PD-L1 inhibitor) and a CTLA4 inhibitor to treat a tumor that is not readily accessible by intratumoral administration. Compound A can be administered intratumorally in a tissue that is remote from the inaccessible tumor in order to induce an abscopal effect. In such cases, the tumor receiving the intratumorally administered dose of Compound A can be a primary tumor or a secondary tumor.

In embodiments where Compound A is administered in combination with an immune checkpoint inhibitor, such as a PD-1 inhibitor, a PD-L1 inhibitor, and/or a CTLA4 inhibitor, the immune checkpoint inhibitor(s) can be administered systemically. For instance, the immune checkpoint inhibitor(s) can be administered intravenously, subcutaneously or intramuscularly. In one embodiment, Compound A is administered intratumorally and a PD-1 inhibitor is administered systemically. In another embodiment, Compound A is administered intratumorally and a PD-L1 inhibitor is administered systemically. In another embodiment, Compound A is administered intratumorally and a CTLA4 inhibitor is administered systemically. In another embodiment, Compound A is administered intratumorally and both a PD-1 inhibitor and a CTLA4 inhibitor are administered systemically. In another embodiment, Compound A is administered intratumorally and a CTLA4 inhibitor is administered systemically. In another embodiment, Compound A is administered intratumorally and both a PD-L1 inhibitor and a CTLA4 inhibitor are administered systemically.

### 5.3. Dosing Regimens

The dosing regimens disclosed herein are capable of evoking a powerful anti-tumor effect without or with significantly reduced concurrent side effects often associated with excessive cytokine production. It has been found that Compound A is capable of eliciting the production of cytokines in a dose dependent manner. Compound A exhibits a profound anti-tumor effect, even at very low levels of cytokine production. For instance, Compound A can be administered safely to cancer patients and provide therapeutic benefits when administered in the range of 1-100 µg/kg. In particular embodiments, Compound A can be administered in the range of 1-50 µg/kg. For instance, Compound A can be administered to a cancer patient in the range of 1-10 µg/kg, 5-10 µg/kg, 5-20 µg/kg, 5-30 µg/kg, 5-40 µg/kg, 5-50 µg/kg, 10-20 µg/kg, 10-30 µg/kg, 10-40 µg/kg, 10-50 µg/kg, 15-20 µg/kg, 15-40 µg/kg, 20-30 µg/kg, 20-40 µg/kg, 20-50 µg/kg, 30-40 µg/kg, 30-50 µg/kg, 5-75 µg/kg, 10-75 µg/kg, 15-75 µg/kg, 20-75 µg/kg, 25-75 µg/kg, 35-75 µg/kg, 5-100 µg/kg, 10-100 µg/kg, 15-100 µg/kg, 20-100 µg/kg, 25-100 µg/kg, 35-100 µg/kg, or 50-100 µg/kg.

In some embodiments, Compound A can be administered to a cancer patient at a dose, e.g., a single or divided doses, in the range of 10-6,500 µg, such as 50-6,500 µg.In particular embodiments, Compound A can be administered to a cancer patient at a dosage, e.g., a single or divided doses, in the range of 100-3,000 µg.In other embodiments, Compound A can be administered to a cancer patient at a dosage e.g., a single or divided doses, in the range of 100-1,200 µg. For instance, Compound A can be administered to a cancer patient in the range of 10-50 µg, 10-100 µg, 10-200 µg, 50-200 µg, 100-200 µg, 100-400 µg, 100-500 µg, 100-800 µg, 200-400 µg, 400-600 µg, 400-800 µg, 100-1,000 µg, 250-1,000 µg, 500-1,000 µg, 500-3,000 µg, 1,000-3,000 µg, 500-4,500 µg, 1,000-4,500 µg, 500-6,500 µg, 1,000-6,500 µg, 2,000-6,500 µg, 3,000-6,500 µg, or 4,500-6,500 µg.

The disclosure provides particular dosing cycles that can maximize efficacy against primary tumors and metastasizing tumors, while ensuring that excessive cytokine production does not compromise the safety of the patient. In one embodiment, the disclosure provides methods of treating a cancer patient by administering particular dosing cycles of Compound A to the patient. In one embodiment, the dosing cycle comprises administering Compound A on days 1, 8, and 15 of a four-week period. This dosing schedule requires administration of Compound A once weekly for three weeks. The patient will not be administered Compound A in the fourth week of the dosing schedule. Subsequent cycles can rely on the same dosing schedule or on different dosing schedules as described below.

In another embodiment, the dosing cycle comprises administering Compound A on says 1 and 15 (i.e., biweekly) of a four-week dosing schedule. Subsequent cycles can rely on the same dosing schedule or on different dosing schedules.

Compound A can be administered to the patient using more than one dosing schedule. For instance the disclosure provides methods of treating a cancer patient by administering cycles of Compound A to the patient, wherein the first cycle comprises administering Compound A on days 1, 8, and 15 of a four-week period and subsequent cycles comprise administering Compound A on days 1 and 15 (i.e., biweekly) of a four-week period. The individual dosages of Compound A administered in the first cycle may be the same or different than the dosages administered in subsequent cycles. For instance, the individual dose administered in the first cycle may be less than doses administered in subsequent cycles.

In another embodiment, the disclosure provides methods of treating cancer, comprising administering to a cancer patient a dosing regimen comprising one or more priming doses of Compound A to the patient at the onset of the therapy, followed by administration of maintenance doses of Compound A. A priming dose refers to a dose that is administered at lower doses than the maintenance doses to increase the tolerance of the body for a particular active agent (e.g., Compound A). It has been found that administration of a priming dose of Compound A improves the safety profile of the compound and allows the compound to be delivered at higher maintenance dosage levels than would otherwise be tolerated. In general, the priming dosage amount will be less than the maintenance doses over the course of a given dosing cycle.

In some embodiments, the priming dose can be administered in a quantity (by weight) that is 2- to 100-fold less than the individual maintenance doses in a given dosing cycle. For instance, the priming dose can be administered in a quantity that is 2- to 70-fold less than, 2-to 50-fold less than, 2- to 30-fold less than, 2- to 20-fold less than, 2- to 10-fold less than, 10-to 50-fold less than, 10- to 30-fold less than, 10- to 20-fold less, or 20- to 30-fold less than the maintenance doses in a given cycle. In some embodiments, the priming dose can be administered in a quantity that is 2- to 4-fold less than the maintenance doses in a given cycle. In some embodiments, the priming dose can be administered in a quantity that is 2- to 5-fold less than the maintenance doses in a given cycle. In some embodiments, the priming dose can be administered in a quantity that is 2- to 8-fold less than the maintenance doses in a given cycle. In some embodiments, the priming dose can be administered in a quantity that is 3- to 5-fold less than the maintenance doses in a given cycle. In some embodiments, the priming dose can be administered in a quantity that is 3- to 8-fold less than the maintenance doses in a given cycle. In some embodiments, the priming dose can be administered in a quantity that is 4- to 8-fold less than the maintenance doses in a given cycle.

In some embodiments, the priming dose can be delivered at a dose that is about 2-fold less than the maintenance doses over the course of a dosing cycle. In some embodiments, the priming dose can be delivered at a dose that is about 3-fold less than the maintenance doses over the course of a dosing cycle. In some embodiments, the priming dose can be delivered at a dose that is about 4-fold less than the maintenance doses over the course of a dosing cycle. In some embodiments, the priming dose can be delivered at a dose that is about 5-fold less than the maintenance doses over the course of a dosing cycle. In some embodiments, the priming dose can be delivered at a dose that is about 10-fold less than the maintenance doses over the course of a dosing cycle. In some embodiments, the priming dose can be delivered at a dose that is about 15-fold less than the maintenance doses over the course of a dosing cycle. In some embodiments, the priming dose can be delivered at a dose that is about 20-fold less than the maintenance doses over the course of a dosing cycle. In some embodiments, the priming dose can be delivered at a dose that is about 50-fold less than the maintenance doses over the course of a dosing cycle. In some embodiments, the priming dose can be delivered at a dose that is about 100-fold less than the maintenance doses over the course of a dosing cycle.

It should be understood that the above relative amounts of priming dose to the individual maintenance doses can be expressed as a ratio. For instance, in an embodiment where the priming dose is administered at a dose that is about 2-fold less than the maintenance doses, a dosing regimen that involves a 1:2 ratio of priming dose to individual maintenance doses is described. Accordingly, in certain embodiments, the present disclosure provides a method of treating cancer comprising administering Compound A to a patient in need thereof according to a dosing regimen that includes a 1:2 to 1:100 ratio of priming dose to individual maintenance doses, such as a ratio of 1:2, 2:5, 3:8, 1:3, 2:7, 1:4, 1:5, 1:6, 1:8, 1:9, 1:10, 1:11, 1:12, 1:15, 1:20, 1:30, 1:50, 1:75, or 1:100, including ranges created by these ratios, such as 1:2 to 1:3, 1:2 to 1:4, 1:2 to 1:5, 1:2 to 1:8, 1:2 to 1:10, 1:4 to 1:8, 1:4 to 1:10, 1:4 to 1:15, 1:4 to 1:20, 1:8 to 1:10, 1:8 to 1:15, 1:8 to 1:20, 1:8 to 1:30, 1:10 to 1:15, 1:10 to 1:20, 1:10 to 1:30, 1:10 to 1:50, 1:20 to 1:30, 1:20 to 1:50, 1:20 to 1:75, 1:20 to 1:100, 1:30: to 1:50, 1:30 to 1:75, 1:30 to 1:100, 1:50 to 1:75, 1:50 to 1:100, or 1:75 to 1:100.

In some embodiments, the present disclosure provides a method of treating cancer comprising administering Compound A to a patient in need thereof according to a dosing regimen that includes a 1:4 or 1:5 ratio of priming dose to individual maintenance doses, or a ratio in the range of 1:3 to 1:6, such as 1:3 to 1:5, 1:4 to 1:6, or 1:4 to 1:5. In other embodiments, the ratio is 1:8 or 1:10, or a ratio in the range of 1:5 to 1:15, such as 1:6 to 1:12, 1:8 to 1:12, 1:8 to 1:10, or 1:9 to 1:10.

In some embodiments, the priming dose of Compound A can be administered to a cancer patient at a dosage in the range of 10-1,000 µg. For instance, the priming dose of Compound A can be administered to a cancer patient in the range of 10-20 µg, 10-40 µg, 10-50 µg, 10-80 µg, 20-40 µg, 40-60 µg, 40-80 µg, 50-100 µg, 100-200 µg, 100-300 µg, 100-500 µg, 200-500 µg, 200-800 µg, 200-1,000 µg, 500-800 µg, or 500-1,000 µg.

In certain embodiments, the priming dose of Compound A can be administered to a cancer patient at a dosage in the range of 0. 15-20 µg/kg, such as 0.15-1 µg/kg, 0.25-1 µg/kg, 0.5-1 µg/kg, 0.5-2 µg/kg, 1-3 µg/kg, 1-5 µg/kg, 2-5 µg/kg, 2-7 µg/kg, 1-10 µg/kg, 2-10 µg/kg, 3-10 µg/kg, 5-10 µg/kg, 5-15 µg/kg, 10-20 µg/kg, or 15-20 µg/kg.

In some embodiments, the maintenance doses of Compound A can be administered to a cancer patient at a dosage in the range of 50-6,500 µg.In particular embodiments, the maintenance doses of Compound A can be administered to a cancer patient at a dosage in the range of 100-3,000 µg.In other embodiments, the maintenance doses of Compound A can be administered to a cancer patient at a dosage in the range of 100-1,200 µg.For instance, the maintenance doses of Compound A can be administered to a cancer patient in the range of 50-200 µg, 100-200 µg, 100-400 µg, 100-500 µg, 100-800 µg, 100-1,000 µg, 200-400 µg, 200-800 µg, 200-1,200 µg, 250-1,000 µg, 400-600 µg, 400-800 µg, 400-1,200 µg, 500-1,000 µg, 500-1,200 µg, 500-1,500 µg, 500-2,000 µg, 500-4,500 µg, 800-1,200 µg, 800-1,500 µg, 800-2,000 µg 1,000-2,000 µg, 1,000-3,000 µg, 1,000-4,500 µg, 2,000-4,500 µg, 500-6,500 µg, 1,000-6,500 µg, 1,500-6,500 µg, 2,000-6,500 µg, or 3,000-6,500 µg.

In certain embodiments, the maintenance doses of Compound A can be administered to a cancer patient at a dosage in the range of 1-100 µg/kg, such as 1-50 µg/kg. For instance, the maintenance doses of Compound A can be administered to a cancer patient in the range of 1-10 µg/kg, 5-10 µg/kg, 5-20 µg/kg, 5-30 µg/kg, 5-40 µg/kg, 5-50 µg/kg, 10-20 µg/kg, 10-30 µg/kg, 10-40 µg/kg, 10-50 µg/kg, 15-20 µg/kg, 15-40 µg/kg, 20-30 µg/kg, 20-40 µg/kg, 20-50 µg/kg, 30-40 µg/kg, 30-50 µg/kg, 5-75 µg/kg, 10-75 µg/kg, 15-75 µg/kg, 20-75 µg/kg, 25-75 µg/kg, 35-75 µg/kg, 5-100 µg/kg, 10-100 µg/kg, 15-100 µg/kg, 20-100 µg/kg, 25-100 µg/kg, 35-100 µg/kg, or 50-100 µg/kg.

In some embodiments, the priming dose can be administered on day 1 of a treatment cycle and the maintenance doses can be administered thereafter at a dosing schedule as described above. The first maintenance dose can be administered at least 2 days following the administration of the priming dose, i.e., on day 3. For instance, the first maintenance dose can be administered 2, 3 4, 5, 6, 7, 8, 9, or 10 days following administration of the priming dose.

In one embodiment, the dosing cycle comprises administering a priming dose of Compound A on day 1 of a treatment cycle followed by administering maintenance doses Compound A on days 8, 15 and 22 (i.e., the first day of weeks 2, 3 and 4) of the treatment cycle, followed by a period of one week (i.e., week 5) where Compound A is not administered to the patient. The maintenance dosing cycle can be repeated or a modified maintenance dosing schedule can be employed.

In another embodiment, the dosing cycle comprises administering a priming dose of Compound A on day 1 of a treatment cycle followed by administering maintenance doses Compound A on days 8 and 22 of the dosing schedule (i.e., biweekly dosing). The maintenance dosing cycle can be repeated or a modified maintenance dosing schedule can be employed.

In another embodiment, the dosing cycle comprises administering a priming dose of Compound A on day 1 of a treatment cycle followed by administering Compound A under two maintenance dosing regimens. The first maintenance dosing regimen comprises administering maintenance doses Compound A on days 8, 15 and 22 (i.e., the first day of weeks 2, 3 and 4) of the treatment cycle, followed by a period of one week (i.e., week 5) where Compound A is not administered to the patient. The second maintenance dosing regimen comprises administering Compound A on a biweekly dosing regimen. For instance, Compound A can be administered at the beginning of weeks 6 and 8 of the dosing cycle. In some embodiments, additional biweekly dosing of Compound A can be administered to the patient. For instance, Compound A can be administered at week 10 of the dosing cycle, weeks 10 and 12 of the dosing cycle, weeks 10, 12, and 14 of the dosing cycle, weeks 10, 12, 14, and 16 of the dosing cycle, and so on.

In some embodiments where a priming dose and maintenance doses of Compound A are administered in accordance with the disclosure, an immune checkpoint inhibitor such as a PD-L1 inhibitor, a PD-1 inhibitor, or a CTLA-4 inhibitor or a combination thereof can also be administered to the patient. The immune checkpoint inhibitor can be administered prior to the priming dose of Compound A or after the priming dose of Compound A. In certain embodiments, the immune checkpoint inhibitor is administered after one full cycle of the maintenance dose has been administered to the patient.

### 6. EXAMPLES

### Example 1. Pharmacokinetic Studies on Compound A

The nonclinical pharmacokinetics (PK) of subcutaneously, intravenously, or intratumorally administered Compound A was investigated in mice at the doses of 0.1 and/or 0.5 mg/kg. Following subcutaneous injection of 0.5 mg/kg of Compound A to male mice, the bioavailability (F) value for Compound A was 1.21, based on the subcutaneous AUC0-t as compared to the intravenous AUC0-t, indicating that Compound A was highly bioavailable. Moreover, Compound A was rapidly cleared for the systemic compartment at a clearance (CL) of 1010 mL/h/kg, a t½ of approximately 15 minutes, and a volume of distribution (Vss) of 255 mL/kg. Compound A was also highly bioavailable in plasma following intratumoral administration in female B16F10 tumor bearing mice, with Frel values of 105% and 112% at doses of 0.1 and 0.5 mg/kg, respectively. This suggests that the abscopal efficacy observed in one of the pharmacology studies might be related to the systemic availability and a direct effect of Compound A on a distant tumor. The evaluations of protein binding and metabolic stability of Compound A in in vitro systems of human, rat, mouse, dog, and monkey showed a 48.9% protein binding in human and a half-life of more than 120 minutes, indicating limited metabolism by CYP450 system.

### Example 2. Toxicological Studies on Compound A

The toxicological evaluations of Compound A included studies in the rat and cynomolgus monkey (non-human primate [NHP]) and should be relevant to assessing potential human risk, as the STING pathway, the target of Compound A, is conserved across species.

The non-good laboratory practice (GLP) studies in the rat and NHP covered a broad range of doses and regimens. The objective of these studies was to evaluate the effects of Compound A following multiple ascending doses, single doses, and multiple doses.

The general findings in these studies were related to the mechanism of action (MOA) of Compound A and can be categorized as inflammatory responses characterized by dose-related increases in STING-dependent gene products including Type 1 Interferons (IFNs) and pro-inflammatory cytokines. The findings were similar in the rat and NHP. The most consistent dose related findings were increases in IFNα, TNFα, and interleukin (IL)-6 in the rat and monkey; IL-8 in the rat; IL-1ra, IFNγ-inducible Protein 10 (IP-10), and Monocyte Chemoattractant Protein 1 (MCP-1) in the monkey. The cytokine levels showed increases in the first 3-6 hours with return to baseline in most cases by 6-24 hours. The innate immune response is characterized by a self-regulating/modulation of the production of these factors. At low doses, the findings would be characterized as pharmacological changes related to the MOA of Compound A. With increasing doses, the changes could be characterized as “exaggerated” pharmacology, which is generally defined as expected changes related to the MOA of Compound A, but greater than the responses needed to affect a therapeutic response. At the highest dose levels, Compound A resulted in toxicity. Mortality was seen in the GLP rat study in the high doses group at 10 and 30 mg/kg. Mortality was also seen at the 3.0 mg/kg dose level. The deaths were attributed to pulmonary edema consistent with Compound A-mediated inflammatory response that was well beyond exaggerated pharmacology and caused the severe toxicity.

The other common findings across species and across the dose ranges/regimens were dose-related and included a range of macroscopic, hematological, clinical chemistry, and microscopic changes. At the low doses, these changes were consistent with a desired therapeutic effect; at the highest doses there were severe toxicities and mortality. In studies with recovery animals, there was either full recovery or a trend to recovery in the parameters showing dose-related changes.

The toxicokinetic findings showed no sex related differences, dose proportionality, and no accumulation in the multiple dose studies.

Moribundity and death was seen at the mid and high dose levels in the rat study. Although there was no clear cause of death, the finding was consistent with a dose-related inflammatory response, due to exaggerated pharmacology of the MOA of Compound A. The 1000 to 3000-fold dose level margins vs. the first-in-human (FIH) dose at the 10 and 30 mg/kg dose levels in the GLP rat study should reduce safety concerns related to the deaths seen in that study.

### Example 3. Cytokine Production Induced by Compound A in Mice

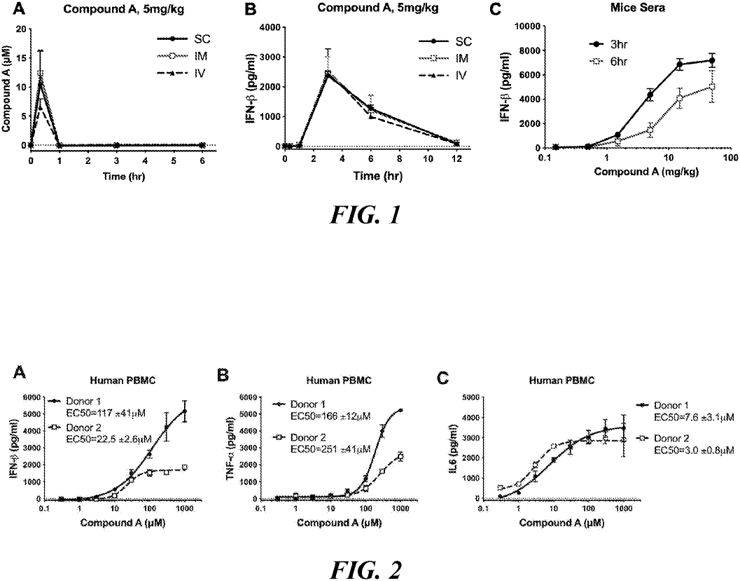

The pharmacokinetic/pharmacodynamic properties of Compound A were evaluated in C57BL/6 mice following dosing by subcutaneous, intramuscular, or intravenous routes of administration. Blood was collected post-dosing for the measurement of Compound A and cytokine levels in plasma. Compound A was detected 15 minutes after injection, in the range of 5-15 µM, and decreased to levels below the limit of detection one hour after administration. The Cₘₐₓ of Compound A was not significantly different between administration routes (FIG. 1, Panel A). High levels of IFNβ in blood were detected 3 hours post dosing, and slowly decreased over time; at 12 hours post dosing, IFNβ levels were near the BLOQ (FIG. 1, Panel B). When mice were injected subcutaneously with serial doses of Compound A (0, 0.15, 0.50, 1.50, 5, 15, and 50 mg/kg), dose-dependent induction of IFNβ (FIG. 1, Panel C) and other cytokines at 3 hours and 6 hours post injection were observed. Notably, 0.50 mg/kg of Compound A induced very low levels of cytokines, however, at doses far lower than 0.50 mg/kg, Compound A already exhibited significant therapeutic effects in various tumor models. For example, 0.005 mg/kg of Compound A was sufficient to inhibit tumor growth and prolong survival in a B16 melanoma model (see Example 6 and FIG. 3), indicating Compound A at therapeutic doses will not cause systemic cytokine problems.

### Example 4. Cytokine Production Induced by Compound A in Peripheral Blood Mononuclear Cells (PBMCs) From Humans

PBMCs from humans were stimulated with serial dilutions of Compound A, and levels of interferon and inflammatory cytokines were measured. The results are shown in FIG. 2, Panels A-C.

### Example 5. Intratumoral and Subcutaneous Administration of Compound A

When administered intratumorally in a tumor bearing mouse, Compound A was highly effective in inhibiting tumor growth in several syngeneic tumor models, including B16F10 (melanoma), MC38 (colon), 4T1 (breast), LL2 (lung) and AG104 (fibrosarcoma). Note that several of these tumors such as B16F10, 4T1, LL2 and AG104 are known to be refractory to antibodies against PD-1, PD-L1, or CTLA-4. In the B16F10 tumor model, twice weekly injection of Compound A for two weeks was efficacious in a dose-dependent manner in the range from 0.1 µg to 10 µg (0.005-0.5 mg/kg). Subcutaneous administration of Compound A was also effective in the MC38 and B16F10 tumor models, but higher doses in the range from 3 µg to 30 µg (0.15-1.5 mg/kg) were required.

Following subcutaneous or intratumoral administration to mice, Compound A was detectable in circulation at 15 minutes. Levels of Compound A administered by these routes or by intravenous administration were below the limit of quantitation (BLOQ) by 1 hour. Compound A induced dose-dependent cytokine production was seen in 3-6 hours following administration, and decreased to BLOQ within 12 hours. At dose levels that showed significant antitumor efficacy, Compound A only induced low levels of cytokine.

In multiple syngeneic tumor models, the combination of a PD-L1 antibody with Compound A showed higher efficacy as compared to Compound A alone at each dose level tested. The administration of Compound A restored responsiveness to PD-L1 antibody in several types of syngeneic tumors which were otherwise refractory to immune checkpoint inhibitors. Several of these studies are described in the examples below.

### Example 6. Effect of Intratumoral Administration of Compound A on B16 Melanoma (Monotherapy and Combination Therapy)

Anti-tumor efficacy studies for Compound A were conducted in syngeneic tumor models in immune competent mice. In a B16 melanoma model, intratumoral administration of Compound A showed dose-dependent suppression of tumor growth and prolonged survival (as compared to controls).

To assess therapeutic activity of Compound A, C57BL/6 immune competent mice received subcutaneous implants of B16F10 melanoma cells on the right flank. Five days later, when tumor volumes were between 50 and 100 mm³, the mice were intratumorally dosed with 0.1, 0.3, or 1.0 µg (equivalent to 0.005, 0.015, or 0.05 mg/kg) of Compound A alone, or in combination with 200 µg of PD-L1 antibody (clone 10F.9G2 available from Bio-X-Cell (catalog # BE0101)). Dosing was repeated every 3-4 days for 4 total doses. Decreased tumor growth and prolonged survival were noted at the dose as low as 0.005 mg/kg (0.1 µg/mice). FIG. 3, Panels A-C show that administration of Compound A significantly reduced tumor volume at all dose levels. The tumor shrinking effect was enhanced when Compound A was administered together with a PD-L1 antibody, even though the PD-L1 antibody showed very low efficacy relative to the control (mock) when administered by itself. FIG. 3, Panels E-G show that the administration of Compound A significantly increased the survival time of the mice relative to control. Once again, the effect was enhanced when Compound A was administered together with the PD-L1 antibody.

### Example 7. Therapeutic Efficacy of Compound A in PD-L1 Antibody-Resistant Tumor Models

Compound A was evaluated alone and in combination with 200 µg of the PD-L1 antibody used in Example 6 in immune checkpoint blockade-resistant tumors. In these studies, mice were implanted with LL2 (lung cancer), 4T1 (breast cancer), AG104Ld (fibrosarcoma), and AG104A (fibrosarcoma) tumors. Following implantation, the animals were dosed intratumorally or subcutaneously with 0.5 mg/kg of Compound A alone or in combination with the PD-L1 antibody, as shown in the table below:

| Tumor Cell Line | Tumor Type | Mice Strain | Dose and Route | Dosing days |
|---|---|---|---|---|
| LL2 | Lung cancer | C57BL/6 | 0.5 mg/kg, IT | 5, 10, 14, 17 |
| 4T1 | Breast Cancer | BALB/c | 0.5 mg/kg, IT | 5, 8, 11, 15 |
| AG104A | Fibrosarcoma | B6C3F1 | 0.5 mg/kg, SC | 4, 7, 10, 14, 17, 21 |
| AG104LD | Fibrosarcoma | B6C3F1 | 0.5 mg/kg, SC | 4, 7, 11, 14, 18, 21 |

IT: intratumoral; PDL1: Programmed Death-Ligand 1; SC: subcutaneous

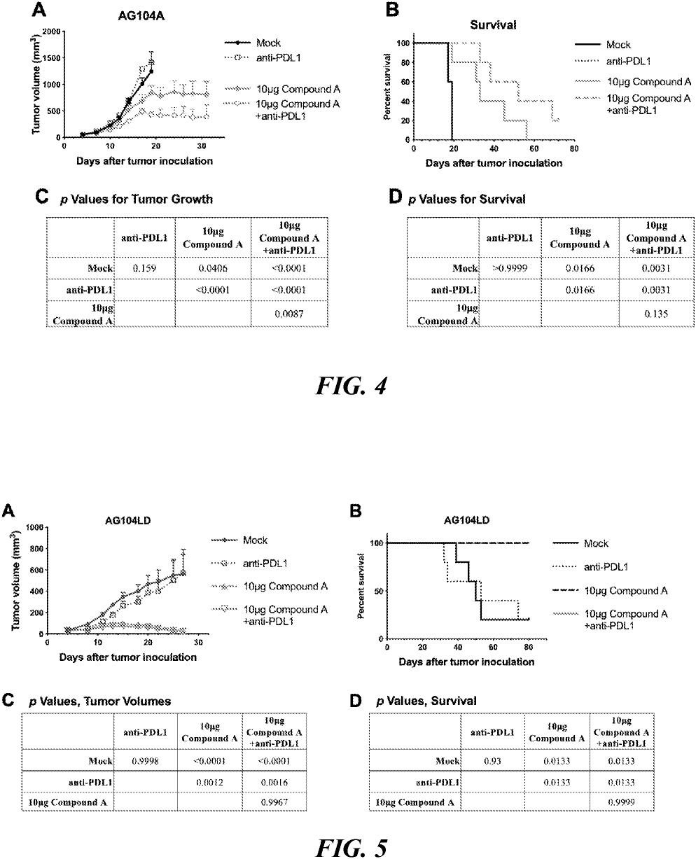

The PD-L1 antibody alone showed no efficacy. In contrast, Compound A when administered alone suppressed tumor growth and prolonged mouth survival in all tested tumor models. Notably, in the AG104A (fibrosarcoma) tumor model, the combination treatment showed improved efficacy as compared to Compound A administered alone, showing that Compound A synergized with the PD-L1 antibody. Representative data for the AG104A, AG104LD, LL2 and 4T1 cell lines are shown in FIGS. 4-7, respectively.

### Example 8. Abscopal Effect of Compound A

An abscopal effect is an immune-mediated phenomenon wherein direct treatment of a primary tumor can lead to a response in a distant tumor. The potential abscopal effect of Compound A was evaluated in B16 melanoma and AG104A fibrosarcoma tumor models evaluating mice bearing tumors on both right flank (primary tumor) and left flank (secondary tumor). In both models, Compound A was administered intratumorally into the primary tumor on days 5, 8, 11 and 15 after tumor inoculation. The secondary tumor was not treated. At doses of 0.15 mg/kg (or 3 µg/mouse) and 0.5 mg/kg (or 10 µg/mouse), tumor growth was suppressed in both primary and distant tumors, and survival was significantly prolonged.

Data from the B16 melanoma and AG104A fibrosarcoma models are shown in FIGS. 8 and 9, respectively. FIG. 8, Panels A-B show that in the B16 melanoma model, the tumor volume of both the primary and distant tumors are significantly decreased following the administration of Compound A relative to the control. Likewise, FIG. 9, Panels A-B show that in the AG104A fibrosarcoma model, the tumor volume of both the primary and distant tumors are significantly decreased following the administration of Compound A relative to the control. Panels A-B of FIG. 9 further show that the combination of Compound A and the PD-L1 antibody (200 µg of the antibody used in Example 6) provide a significant synergistic increase in the reduction of the tumor volume of both primary and distant tumors versus Compound A or the PD-L1 antibody alone. This effect is synergistic and not merely additive because the PD-L1 antibody had no effect on tumor volume when administered in the absence of Compound A. Panel E of FIG. 9 shows that administration of Compound A increases the survival of the tumor-bearing mice, the extent of which is also synergistically enhanced by combination with the PD-L1 antibody, which again had no effect in the absence of Compound A.

### Example 9. Effect of Compound A on Tumor Metastasis

A B16 melanoma lung metastasis model was used to evaluate the effect of Compound A on tumor metastasis. Compound A was administered intratumorally on days 5, 8, and 11 after tumor inoculation alone or in combination with a PD-L1 antibody (200 µg of the antibody used in Example 6). As shown in FIG. 10, Panel A, administration of Compound A either alone or in combination with the PD-L1 antibody resulted in significant reduction in tumor volume. As depicted in FIG. 10, Panel C, images showing lung metastases show that the number of metastases in the lung is dramatically reduced following administration of Compound A, either alone or in combination with the PD-L1 antibody.

### Example 10. Administering a Priming Dose of Compound A

Male and female cynomolgus monkeys were assigned to groups and doses of Compound A were administered. Animals were dosed via subcutaneous injection at a volume of 2 mL/kg. The vehicle control article/diluent was phosphate-buffered saline (PBS).

Escalation of Compound A dose levels was tolerated up to 3.0 mg/kg/dose, with findings limited to increased body temperature and elevated IFNα, IL-6, and TNFα cytokine levels. IFNα, TNFα, and IL-6 levels were measured at 3, 6, and 12 hours post-dosing. Dose related but variable changes were observed. Moderate levels of IFNα were noted in the 1 mg/kg and 3 mg/kg groups at 3 hours and 6 hours post dosing. Higher levels of IFNα were seen in the 10 mg/kg group. IFNα levels at 3 mg/kg and 10 mg/kg decreased 12 hours after dosing, but did not return to pre-dose levels. Increases in plasma IL-6 levels were noted at 3 and 6 hours post dosing in all groups. IL-6 increases at 3 mg/kg and 10 mg/kg persisted at 12 hours postdose. TNFα levels increased at 3 hours in the 1 mg/kg group. Lower levels of TNFα were observed in the 3 mg/kg and 10 mg/kg groups. The cytokine responses are consistent with the predicted STING pathway activation. Morbidity was observed within 1 day of administration of the 10 mg/kg/dose; as such, 3 mg/kg was selected as the high dose for the following repeat-dose phase (Phase II).

In Phase II, 3 weekly administrations of 0.3 mg/kg of Compound A were tolerated. The 3 mg/kg dose in naïve animals was not tolerated and led to clinical observations of morbidity or death within 1 day of dosing. The findings were consistent with Compound A-mediated inflammatory response that was considered the probable cause of death. At the 3 mg/kg dose level, compound-related dose-dependent increases in plasma IL-1ra, IL-6, and IFNα cytokine levels were generally noted at 3 and 6 hours with levels returning to those noted in controls for IL-6 and IFNα. There were sporadic increases in IL-12, granulocyte-colony stimulating factor (G-CSF), and IFNγ levels. These changes, however, were generally inconsistent between sexes, not dose-dependent, and of a small magnitude and, hence, considered only potentially related to Compound A. Changes in levels of pro-inflammatory cytokines and chemokines MCP-1 and IP-10 were suggestive of an inflammatory response with resolution by 24 hours postdose. Exposure, as assessed by Compound A mean Cmax, AUC₀₋₂, AUC₀₋₈, and AUC₀₋₂₄ values, generally increased with the increase in dose level from 0.3 to 3 mg/kg/day on Day 1 of Phase II, and were generally dose-proportional. No accumulation of Compound A was observed after multiple doses of 0.3 mg/kg/day in monkeys. In general, sex differences in Compound A mean Cmax, AUC₀₋₂, AUC₀₋₈, and AUC₀₋₂₄ values were less than 2-fold.

During Phase III, all animals administered three weekly doses of 0.6 or 1.0 mg/kg/day of Compound A survived until scheduled sacrifice. A priming dose of 0.1 mg/kg/day was administered 4 days prior to the first dose of 1.0 mg/kg/day Compound A to potentially allow a tolerance to develop to avoid the acute mortality noted during Phase II following administration of 3.0 mg/kg/day of Compound A to naïve animals. When administered at 0.1 mg/kg/day, Compound A did not cause significant increase in plasma IFNα levels in either male or female. Increased plasma levels of IL-6 were noted 3 hours and 6 hours postdose; however, IL-6 levels returned to a non-detectable level 24 hours postdose. Elevated levels of TNFα were noted 6 hours postdose in male and 3 hours and 6 hours postdose in female. In both cases, TNFα levels returned to non-detectable level 24 hours post dosing. Slight elevation of IP-10 was noted 3 hours post dosing in male and female animals. When administered at 0.6 mg/kg/day, Compound A did not cause significant increase in plasma IFNα levels in either male or female. Increased plasma levels of IL-6 were noted 3 hours and 6 hours postdose. Elevated levels of TNFα were noted 6 hours postdose in male and 1.5, 3, and 6 hours postdose in female. No significant elevation of IP-10 was noted throughout the time course. When administered at 1 mg/kg/day, Compound A did not cause significant change in IFNα levels at 1.5 and 3 hours postdose, but elevated levels of this cytokine were observed 6 hours postdose in both male and female. Marked increase in IL-6 levels was noted at 3 and 6 hours postdose in both male and female. Elevated TNFα levels were noted at 1.5, 3, and 6 hours postdose in both male and female. A slightly higher predose level of IP-10 was noted in male only, but no increased IP-10 level was observed 1.5, 3, and 6 hours postdose.

In conclusion, administration of ≥ 3.0 mg/kg of Compound A was not tolerated in naïve animals and led to acute morbidity and/or death, which was attributed to pulmonary edema. Edema is consistent with an inflammatory related pathology and the exaggerated pharmacology of the mode of action of Compound A. Administration of 3 weekly doses of 1.0 mg/kg/day (preceded by a priming dose of 0.1 mg/kg) or 0.6 mg/kg (without a priming dose) was tolerated. Animals tolerated an escalation to 3.0 mg/kg in Phase I, due to previous administrations at lower levels that allowed a tolerance to develop. For animals administered with 0.6 or 1.0 mg/kg/day, compound-related findings were limited to a transient body temperature increase and mild to moderate clinical and anatomic pathology findings.

### Example 11. Triple Combination Therapy

On day 0, female C57BL6 mice (5 in each group) were subcutaneously implanted with 10⁶ of B 16F 10 melanoma cells (ATCC CRL6475) on their flanks. On day 6, tumors were measured and mice were regrouped so that each group had similar average tumor volumes (~70 mm³). On day 6, 10, and 14, mice were mock treated or treated with: 0.3 µg of Compound A intratumorally (I.T.); 50 µg of CTLA4 antibody (BioXcell BE0164, I.T.); or combination of 0.3 µg of Compound A (I.T.) and 200 µg of CTLA4 antibody intraperitoneally (I.P.). In the same set of experiments, the combination of 0.3 µg of Compound A (I.T.) and 200 µg of PD-L1 antibody (I.P.) was also tested with and without the combination of 200 µg of CTLA4 antibody (I.P.). Tumor volumes were measured every 2-3 days and mouse survival was monitored daily.

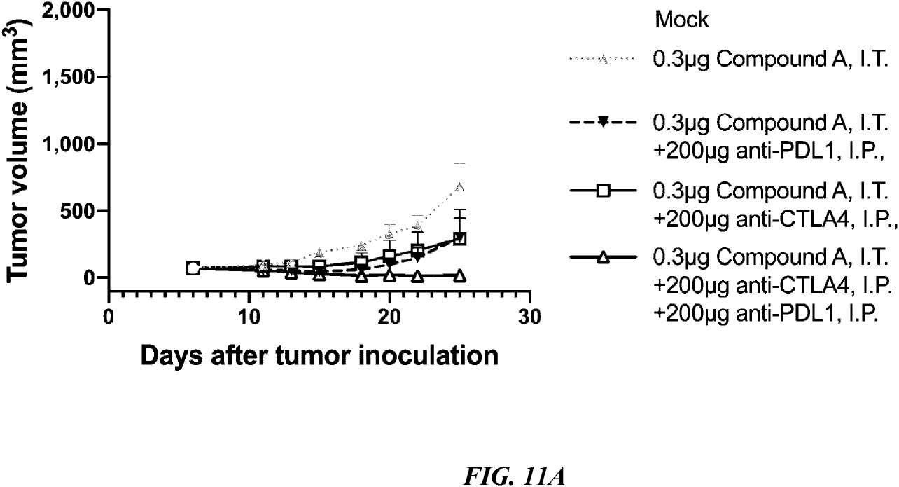

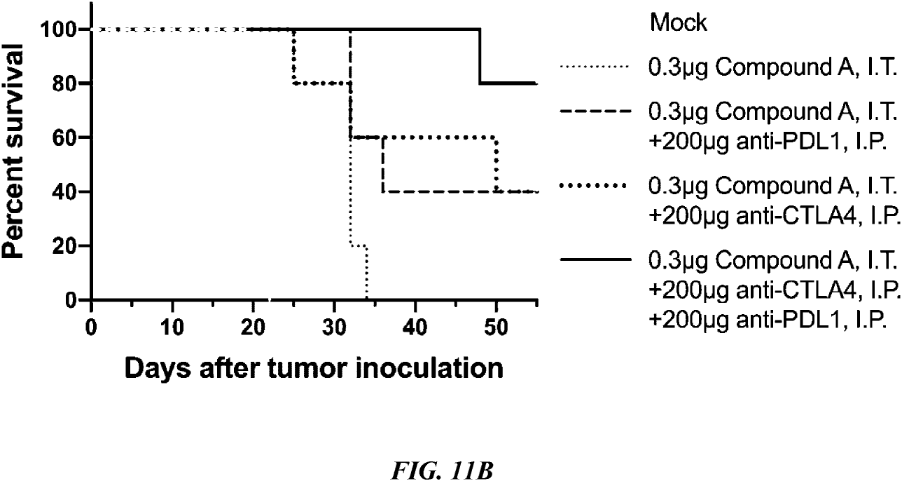

As shown in FIGS. 11A and 11B, each combination therapy had profound effects on tumor volume and overall survival, with the triple combination of Compound A, the PD-L1 antibody (I.P.) and the CTLA4 antibody (I.P.) being the most efficacious.

1. A method of treating a cancer in a patient, comprising administering multiple cycles of a compound to the patient;
wherein the compound has the following structure of or a pharmaceutically acceptable salt thereof: 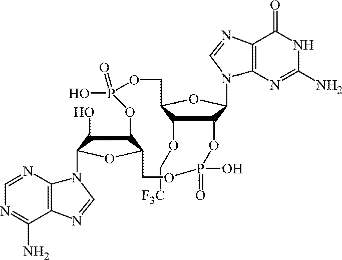
wherein the first cycle comprises administering the compound on days 1, 8, and 15 of a four-week period; and
wherein subsequent cycles comprise administering the compound on days 1 and 15 of a four-week period.

2. The method of claim 1, wherein the compound is administered intratumorally.

3. The method of claim 1, wherein the compound is administered systemically.

4. The method of claim 1, wherein the compound is administered subcutaneously or intramuscularly.

5. The method of any one of claims 1-4, wherein the compound is administered at a dose in the range of 50 µg to 6,500 µg on each day a cycle specifies administering.

6. The method of any one of claims 1-4, wherein the compound is administered at a dose in the range of 100 µg to 3,000 µg on each day a cycle specifies administering.

7. The method of any one of claims 1-4, wherein the compound is administered at a dose in the range of 100 µg to 1,200 µg on each day a cycle specifies administering.

8. The method of any one of claims 1-4, wherein the compound is administered at a dose in the range of 100 µg to 500 µg on each day a cycle specifies administering.

9. The method of any one of claims 1-4, wherein the compound is administered at a dose in the range of 100 µg to 200 µg on each day a cycle specifies administering.

10. The method of any one of claims 1-9, further comprising administering an immune checkpoint inhibitor to the patient.

11. The method of claim 10, wherein the immune checkpoint inhibitor is administered prior to the first cycle.

12. The method of claim 11, wherein the first cycle is initiated after the patient’s cancer has stabilized.

13. The method of claim 12, wherein the patient has undergone at least 1 cycle of immune checkpoint inhibitor therapy prior to the first cycle.

14. The method of claim 10, wherein the immune checkpoint inhibitor is administered after the first cycle.

15. The method of any one of claims 10-14, wherein the immune checkpoint inhibitor is selected from a PD-L1 inhibitor, a PD-1 inhibitor, a CTLA-4 inhibitor, or a combination thereof.

16. The method of claim 15, wherein the immune checkpoint inhibitor is a PD-1 inhibitor.

17. The method of claim 15, wherein the immune checkpoint inhibitor is a PD-L1 inhibitor.

18. The method of claim 15, wherein the immune checkpoint inhibitor is a CTLA-4 inhibitor.

19. A method of treating a cancer in a patient, comprising administering to the patient a compound according to a dosing regimen comprising a priming dose of the compound followed by maintenance doses of the compound;
wherein the compound has the following structure or a pharmaceutically acceptable salt thereof: 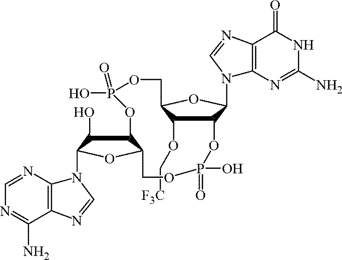
wherein the amount of the compound in the priming dose is less than the amount of the compound in each maintenance dose.

20. The method of claim 19, wherein the patient had not previously been administered the compound prior to administering the priming dose.

21. The method of claim 19 or claim 20, wherein the maintenance doses are administered in cycles, wherein the first cycle comprises administering the compound on days 1, 8, and 15 of a four-week period and subsequent cycles comprise administering the compound on days 1 and 15 of a four-week period.

22. The method of claim 21, wherein the first maintenance dose is administered at least three days after administering the priming dose.

23. The method of claim 21, wherein the first maintenance dose is administered one week after administering the priming dose.

24. The method of any one of claims 19-23, wherein the priming dose is 2- to 100-fold less by weight than each maintenance dose.

25. The method of any one of claims 19-23, wherein the priming dose is 2- to 5-fold less by weight than each maintenance dose.

26. The method of any one of claims 19-23, wherein the priming dose is 5- to 20-fold less by weight than each maintenance dose.

27. The method of any one of claims 19-26, wherein the compound is administered intratumorally.

28. The method of any one of claims 19-26, wherein the compound is administered systemically.

29. The method of any one of claims 19-26, wherein the compound is administered subcutaneously, intravenously, or intramuscularly.

30. The method of any one of claims 19-29, wherein each maintenance dose is in the range of 50 µg to 6,500 µg.

31. The method of any one of claims 19-29, wherein each maintenance dose is in the range of 100 µg to 3,000 µg.

32. The method of any one of claims 19-29, wherein each maintenance dose is in the range of 100 µg to 1,200 µg.

33. The method of any one of claims 19-29, wherein each maintenance dose is in the range of 100 µg to 500 µg.

34. The method of any one of claims 19-29, wherein each maintenance dose is in the range of 100 µg to 200 µg.

35. The method of any one of claims 19-34, further comprising administering an immune checkpoint inhibitor to the patient.

36. The method of claim 35, wherein the immune checkpoint inhibitor is administered prior to administering the priming dose.

37. The method of claim 36, wherein the dosing regimen is initiated after the patient’s cancer has stabilized.

38. The method of claim 37, wherein the patient has undergone at least 1 cycle of immune checkpoint inhibitor therapy prior to administering the compound according to the dosing regimen.

39. The method of claim 35, wherein the immune checkpoint inhibitor is administered after administering the priming dose.

40. The method of any one of claims 35-39, wherein the immune checkpoint inhibitor is a PD-L1 inhibitor.

41. The method of any one of claims 35-39, wherein the immune checkpoint inhibitor is a PD-1 inhibitor.

42. A method of treating a cancer patient who is unresponsive to PD-1 or PD-L1 inhibition, comprising administering to the patient a compound in combination with a PD-1 or a PD-L1 inhibitor;
wherein the compound has the following structure or a pharmaceutically acceptable salt thereof: 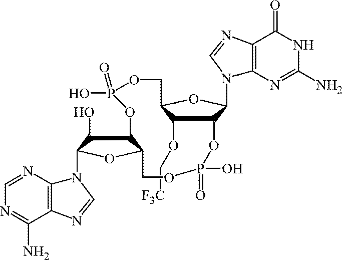
.

43. The method of claim 42, wherein the compound is administered intratumorally.

44. The method of claim 42, wherein the compound is administered systemically.

45. The method of claim 42, wherein the compound is administered subcutaneously or intramuscularly.

46. The method of any one of claims 42-45, wherein the compound is administered at a dose in the range of 10 µg to 6,500 µg.

47. The method of any one of claims 42-45, wherein the compound is administered at a dose in the range of 100 µg to 3,000 µg.

48. The method of any one of claims 42-45, wherein the compound is administered at a dose in the range of 100 µg to 1,200 µg.

49. The method of any one of claims 42-45, wherein the compound is administered at a dose in the range of 100 µg to 500 µg.

50. The method of any one of claims 42-45, wherein the compound is administered at a dose in the range of 100 µg to 200 µg.

51. The method of any one of claims 1-50, wherein the cancer is selected from prostate cancer, pancreatic cancer, lymphoma, head and neck cancer, kidney cancer, melanoma, colon cancer, breast cancer, and lung cancer.

52. The method of any one of claims 1-51, wherein the patient has a metastasizing tumor.

53. A method of treating a cancer in a patient, comprising intratumorally administering to the patient a dose of a compound at a site that is accessible for intratumoral administration;
wherein the compound has the following structure or a pharmaceutically acceptable salt thereof: 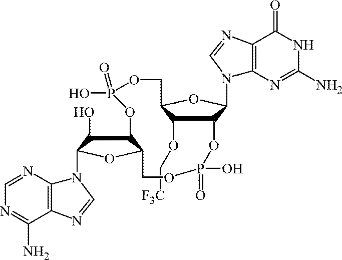
wherein some occurrences of the cancer are inaccessible for intratumoral administration; and
wherein the dose provides sufficient cytokine activation to promote an immune response by the patient against the inaccessible occurrences of the cancer.

54. The method of claim 53, further comprising administering an immune checkpoint inhibitor to the patient.

55. The method of claim 53, wherein the immune checkpoint inhibitor is a PD-L1 inhibitor.

56. The method of claim 53, wherein the immune checkpoint inhibitor is a PD-1 inhibitor.
